(19) United States
(12) Patent Application Publication
(10) Pub. No.: US 2010/0063499 A1
(43) Pub. Date: Mar. 11, 2010

(54) Device for Removing an Endoprosthesis or an Implant that is Implanted in the Body and is Made of a Synthetic Material

(76) Inventors: Andreas Pein, Einhaus (DE); Christian Högfors Ziebell, Schwerin (DE); Matthias Honl, Klagenfurt (AT)

Correspondence Address: GUDRUN E. HUCKETT DRAUDT, SCHUBERTSTR. 15A, WUPPERTAL, 42289, DE

(21) Appl. No.: 12/162,656

(22) PCT Filed: Jan. 31, 2007
(86) PCT No.: PCT/DE07/00211

§ 371 Date: Nov. 18, 2009

(30) Foreign Application Priority Data
Jan. 31, 2006 (DE) 10 2006 005 224.2

(51) Int. Cl. A61B 18/18; A61N 1/30

(52) U.S. Cl. 606/41; 604/20

(57) ABSTRACT

The invention relates to devices for removing an endoprosthesis or an implant which is implanted in the body and is made of a synthetic material. The inventive devices and methods are characterized especially by the fact that a minimal amount of tissue decomposes such that the endoprosthesis or the implant can be removed gently, the endoprosthesis, the implant, or a section thereof embodying a conductor. The endoprosthesis or the implant and at least one electrode that is connected in an electrically conducting manner to a surface area of the endoprosthesis of the implant, or at least one electrode which is in contact with a region of the material surrounding the endoprosthesis or the implant, are connected to an HF generator as an AC current source in order to separate the endoprosthesis or the implant from material surrounding the same in such a way that power is applied to the tissue and/or the biomaterial that surrounds the endoprosthesis or the implant. The endoprosthesis or the implant is coupled to an apparatus in order to be removed.

The invention concerns devices for removing an endoprosthesis or an implant that is implanted in the body and is comprised of synthetic material.

Prostheses or implants introduced into the human body are generally removed at the end of their service life by mechanical methods. This concerns in particular prostheses and implants that are connected with the tissue type bone. In order to be able to remove, for example, an endoprosthesis or a tooth implant from the bone, the gap-like boundary layer between bone and the implanted material that is not easily accessible must be released by mechanical working of the bone by chiseling, drilling, milling or sawing. Such methods constitute massive interventions that destroy the living tissue unnecessarily and therefore cause tissue loss. The possibilities for anchoring a new endoprosthesis or a new implant are thus reduced because as a result of the introduced defects the primary stability is reduced and inevitably a longer healing process is required.

A further disadvantage of mechanical processing resides in the possible destruction of the surrounding tissue as well as in the long duration of the operation that increases morbidity of the operation that is evident by high blood loss and increased infection rate.

In the case of revision surgery, for example, of a hip endoprosthesis, there is the problem of having separate materials at a narrow gap, i.e., the interface between bone and prosthesis. For conventional tools this gap is almost inaccessible. In many cases the bone must therefore be additionally “windowed” which makes more difficult the implantation of a new prosthesis. In case of cemented hip prostheses in general the metallic prosthesis shank can be extracted from the cement tube but cement remaining in the bone also presents the surgeon with great problems.

Hitherto described alternatives with respect to the conventional tools have not found clinical acceptance for various reasons. Neither the extracorporal shockwave lithotrypsy (ESWL) that is to generate micro fractures in the bone cement and reduce shearing force load capacity nor the melting and subsequent removal of the biomaterial bone cement by ultrasound probes nor the laser application has led to a breakthrough.

The invention disclosed in claims 1 and 20 has the object to remove endoprostheses or implants introduced into the body with minimal damage to the tissue so that the patient is not harmed by a tedious surgical procedure and the biological as well as mechanical conditions for a re-implantation remain optimal.

This object is solved by the features disclosed in claims 1 and 20.

The devices and methods for removing an endoprosthesis or an implant that is implanted in the body and comprised of synthetic material are characterized in particular by minimal damage of biological tissue so that a gentle removal is enabled. As is well known in the art, endoprostheses or implants are comprised at least partially of a metal or another non-metallic material with electric conductivity and are thus electric conductors.

The endoprosthesis or the implant as a conductor and either at least one electrode that is electrically conductingly connected to a surface area of the endoprosthesis or the implant or at least one electrode in contact with an area of the material surrounding the endoprosthesis or the implant are connected to an HF generator as an alternating current source. In this way, an energy introduction into the tissue or biomaterial that surrounds the endoprosthesis or the implant is realized and the tissue or biomaterial becomes detached from the endoprosthesis or the implant.

The energy introduction causes in the tissue a protein denaturation in particular in the area of the pulling-resistant collagen fibers so that the tissue that surrounds the endoprosthesis or the implant is weakened and, for example, in the case of bone, becomes brittle. The same thermal damage is also observed in case of a biomaterial, such as bone cement, wherein also the mechanical strength is reduced.

For removal, the endoprosthesis or the implant is coupled to a device simultaneously with or after HF processing.

By utilizing high frequency surgery, in the following referred to simply as HF surgery, the expenditure and tissue damage when detaching endoprostheses or implants is reduced as much as possible. The utilization HF surgery provides a gentle method wherein the destruction of cells is avoided as much as possible and, in this way, the healing process is positively affected. The same holds true also for blood loss during operation that is inhibited as much as possible by application of HF surgery.

By connecting the endoprosthesis or the implant and the electrode with the high frequency generator, in the following referred to simply as HF generator, preferably thermal effects are utilized that are the result of energy introduction into a tissue volume that is as small as possible. The heat output that is produced therein is directly proportional to the specific resistance and the square of the current density in accordance with Joule's law.

In case of minimal HF current acting on the biological tissue only fide heat is generated that is distributed uniformly in the biological tissue. When the power and thus the temperature are increased, a phase of protein denaturation occurs until with increasing power and temperature an increasingly faster evaporation of the extracellular and intracellular liquid is caused. This causes a reduction of the thermal conductivity of the tissue and the electrical resistance will rise so that the separation process is self-limiting. Upon further increase of power and thus temperature, the evaporation process will accelerate until it becomes explosion-like and, as a result of the evaporation heat, so much energy will be consumed that the heated area becomes narrower. This can be continued up to the point of spark formation.

This results in desiccation (drying), coagulation, tissue separation and fulguration (tissue destruction by sparks).

Coagulation effects at a temperature between 50° C. and 100° C. the transition of protein into denatured protein. This transition is associated with a significant loss of liquid. In this way, shrinkage of tissue cells as well as constriction of vessels up to the point of complete closure are effected. At the same time, the thermal conductivity of the tissue is reduced and the electrical resistance increases so that a further propagation of the heat into the depth of the biological tissue is inhibited.

Advantageously, the endoprosthesis, the implant or an area thereof is itself a conductor. Upon contact with at least one electrode and connection to the HF generator as an alternating current source either the surface, or a surface area, between contacted electrodes of the endoprosthesis or the implant itself is heated.

At higher frequencies, the alternating current that is passing through the endoprosthesis, the implant or through an area of the endoprosthesis or the implant does not fill the entire cross-section so that high-frequency alternating currents flow only in a surface layer (this is referred to in physics as skin effect). The current density decreases greatly from the surface toward the interior of the endoprosthesis or the implant. This is the result of the alternating magnetic flux that extends in the interior of the endoprosthesis or the implant generating additional voltages and currents in accordance with the law of induction; they lead to current distribution across the cross-section and, in turn, also to a redistribution of the magnetic flux wherein with increasing frequency the current in the interior of the endoprosthesis or the implant is increasingly weakened and concentrated on the surface. In turn, the magnetic field in the interior of the prosthesis that is caused by the supplied alternating current and is also periodic is surrounded according to the law of induction also by streamlines of the electrical field. These streamlines of the electrical field are oriented in the interior of the prosthesis in a direction opposite to the electrical field that is present therein and reinforces the electrical field at the surface. According to Ohm's law the amount of current density at the surface of the prosthesis is greater than in the interior. In this way, only a surface area of the endoprosthesis or the implant is heated. A complete heating of the endoprosthesis or the implant is advantageously prevented as much as possible.

The obtainable heating of the surface or of a surface area of the endoprosthesis or of the implant increases with the frequency of the alternating current and with the conductivity of the endoprosthesis or the implant and is furthermore dependent on the shape of the cross-section and the action of the neighboring conductors.

The connecting location between endoprosthesis or implant and the material surrounding it is supplied through the connection of the endoprosthesis or the implant and the electrode with the HF generator with a power of preferably 100 to 300 W at a frequency in the Megahertz range. The magnitude of the power depends on the time of the supply action so that greater powers are also possible.

Heating of the endoprosthesis or of the implant can be done also only section-wise wherein this area of the endoprosthesis or the implant is at the same time the conductor. On the one hand, a terminal area of this area is connected either directly or by means of a second electrode and, on the other hand, the electrode as the first electrode is connected to the HF generator.

The alternating current flows only in this area of the endoprosthesis or the implant wherein the heat is also produced in this area only. The area represents a section of the endoprosthesis or of the implant, and heating is realized section-wise sequentially across the entire length of the endoprosthesis or the implant.

In this way, also complex configurations of the endoprostheses or the implants, endoprosthesis or implants with different cross-sectional dimensions, or elongate endoprostheses or implants can be separated section-wise from the surrounding biological and/or biocompatible materials.

Advantageous embodiments of the invention are disclosed in claims 2-19 and 21-25.

In accordance with the further embodiments of claims 2 and 21, the electrode or said electrode and a further electrode is/are guided in a passage or each guided in a passage in the tissue or biomaterial and in this context are preferably arranged at an angle to the surface of the endoprosthesis or the implant. In this connection, the at least one electrode is pressed against the endoprosthesis or the implant so that the electrical connection is based on the contact of the end of the electrode on a surface area of the endoprosthesis or the implant. For a section-wise heating of the endoprosthesis or the implant in a passage that is positioned at a spacing in the longitudinal direction the second electrode is guided so that the spaced apart area of the endoprosthesis or the implant is the conductor.

When in accordance with the further embodiments of claim 3 two passages with a first electrode and a second electrode are used, a section-wise heating of the endoprosthesis or of the implant according to the placement of the heads of the electrode can be realized, wherein the area of the endoprosthesis or the implant inbetween is the conductor. The connection is realized by the heads of the electrodes that, in cross-section, are configured to be greater than the remaining components.

Advantageously, the electrode head of the at least one electrode according to the embodiment of claim 4 comprises a spreading mechanism so that a greatest possible contact surface between the respective head and the endoprosthesis or the implant and thus a reduced transfer resistance is present.

The at least one electrode has according to the embodiment of claim 5 at least one cavity connected to a conveying device for flowable materials. In this way, the electrode or the connecting member can be preferably cooled.

The end area of the at least one electrode has according to the embodiment of claim 6 at least one opening wherein this opening and the at least one cavity are connected to one another. In this way, it is easily possible to supply to the cutting location or surgery location a flowable material that enhances advantageously the surgery.

The opening with the cavity can be utilized also for removing materials that are produced during the operation.

In case of several cavities that are separated from one another, flowable materials can be supplied simultaneously and can be removed alone or enriched with further materials so that a flushing process of the surgery area can be realized.

Advantageously, according to the embodiment of claim 7, the at least one electrode can be at the same time a cutting or a non-cutting tool for the tissue or biomaterial.

The at least one electrode according to the embodiment of claim 8 for introducing a passage into the tissue or biomaterial is at the same time an electrode relative to a preferably plate-shaped electrode that is connected either to said HF generator or an HF generator.

The plate-shaped electrode is connected areally to the patent. In this way, the current density is to be reduced as much as possible in order to avoid damage of the healthy tissue in accordance with Joule's law. In the immediate surroundings of the electrode the current density is highest and therefore the thermal effect strongest. This effect decreases with the square of the distance relative to the electrode. In case of procedures with small current densities a plate-shaped electrode can be omitted wherein the current circuit is closed by the impedance of the human body to ground.

The at least one electrode according to the embodiment of claim 9 for introducing a passage is comprised of two spaced apart partial electrodes that are connected either to said HF generator or an HF generator.

This is advantageously the bipolar application technique of HF surgery. In this connection, the current flows only through that part of the tissue or biomaterial in which the surgical action is desired. Two electrodes that are isolated relative to one another and between which the HF voltage is present are guided to the location where the ablation or cut is to be performed. The electric circuit is closed through the tissue or the biomaterial that is destroyed in this way. As a result of the minimal path length less power is required and the surrounding tissue is not stressed.

The electrode that contacts the at least one area of the tissue or biomaterial that surrounds the endoprosthesis or the implant is a sleeve in accordance with the embodiments of claims 10 and 22. The material surrounding the endo prosthesis or the implant is bone. The sleeve is placed as an electrode about the exposed terminal area of the bone and is contacted therewith in an electrically conducting way. This electrode is for this purpose preferably the reference electrode.

The sleeve according to the embodiment of claim 11 is a component of a cooling device wherein the sleeve is provided with at least one passage for a cooling agent or with Peltier elements. The resulting heat is concentrated onto the area to be separated. Burns are prevented as much as possible.

By twisting wires of small cross-sections together to form at least one electrode according to the embodiment of claim 12, no or only a reduced skin effect in the electrode itself is produced.

In accordance with the embodiment of claim 13, at least the surgery area is partially located within a Faraday cage wherein the latter is connected to a potential. In this way, in particular the electromagnetic disturbance produced by the effects of the HF generator in the surroundings are substantially prevented so that other devices required for the patient's care are not impaired with respect to their function. At the same time, the effects of such interference fields will be lowered with regard to other regions of the patient himself.

The endoprosthesis or the implant as a conductor or at least one area of the material surrounding the endoprosthesis or the implant by means of a further electrode contacting this material and the electrode are connected according to the embodiments of claims 14 and 23 with the HF generator as an alternating current source in such a way that the skin effect is acting in at least one surface area of the endoprosthesis or the implant. The skin effect is characterized advantageously in that the high frequency alternating currents flow only within a thin surface layer. At higher frequencies the alternating current flowing through the endoprosthesis, the implant or though an area of the endoprosthesis or the implant does not fill out the entire cross-section. The skin effect has the effect that the heat is produced only in this surface layer. The surface layer is either the endoprosthesis or the implant itself or only a portion thereof. In the latter case, heating of the endoprosthesis or the implant acts sequentially and section-wise across the entire length. In this way, even complex configurations of endoprostheses/implants, endoprostheses/implants with different cross-sectional dimensions, or elongate endoprostheses/implants can be separated section-wise from the surrounding tissue and/or biomaterial.

The HF generator is connected in accordance with the embodiments of claims 15 and 24 with a data processing device in such a way that the energy introduction through the electrode is realized as a function of the frequency of the high voltage, of the geometry of the prosthesis or the implant, and the total electric power.

The data processing device is connected with the HF generator and in accordance with the embodiment of claim 16 advantageously with a scanner for determining the geometry of the endoprosthesis or the implant and/or adhesion of the endoprosthesis or the implant in/on the tissue and biomaterial as a control or governing circuit wherein the magnitude of energy introduction is controlled as a function of the geometry and/or adhesion between endoprosthesis or implant as well as tissue and biomaterial.

The data processing device is connected with the HF generator and in accordance with the embodiment of claim 17 advantageously with a scanner for determining the geometry of the endoprosthesis or the implant and/or adhesion of the endoprosthesis or the implant in/on the tissue and biomaterial as a control or governing circuit in such a way that the magnitude of energy introduction and the position of the first electrode and the second electrode are controlled and selected as a function of the geometry and/or adhesion between endoprosthesis or implant as well as tissue and biomaterial.

According to the embodiment of claim 18 the tool for removing the endoprosthesis or the implant is a device that transmits tensile forces, pressure forces and/or bending moments onto the endoprosthesis or the implant wherein the detachment is mechanically assisted and the removal of the endoprosthesis or the implant is done mechanically.

According to the embodiment of claim 19, a coil or a permanent magnet are arranged such that the location and the time of snap-over of the high frequency current between tissue or biomaterial and endoprosthesis or implant can be controlled.

According to the embodiment of claim 25, into the tissue or biomaterial that surrounds the endoprosthesis or the implant an electrically conducting liquid is introduced. This is preferably an electrolyte solution. The detachment and removal of the endoprosthesis or the implant is facilitated.

Embodiments of the invention are shown schematically in the drawings and will be explained in the following in more detail.

It is shown in:

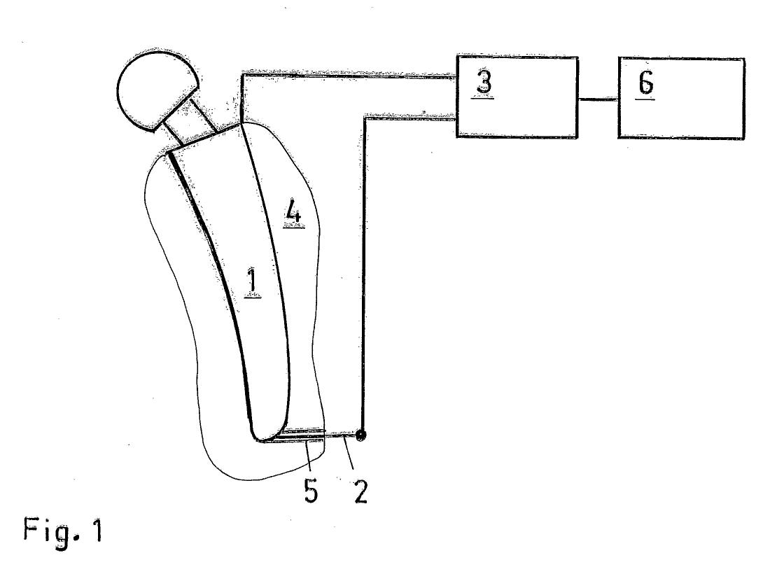

FIG. 1 a device for removing an endoprosthesis with an electrode positioned at an angle to the endoprosthesis;

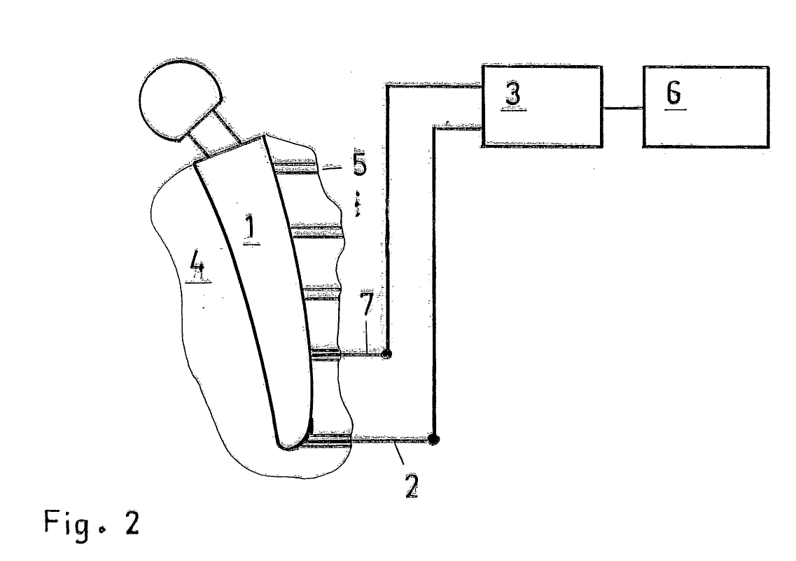

FIG. 2 a device for removing an endoprosthesis with two electrodes positioned at an angle to the endoprosthesis;

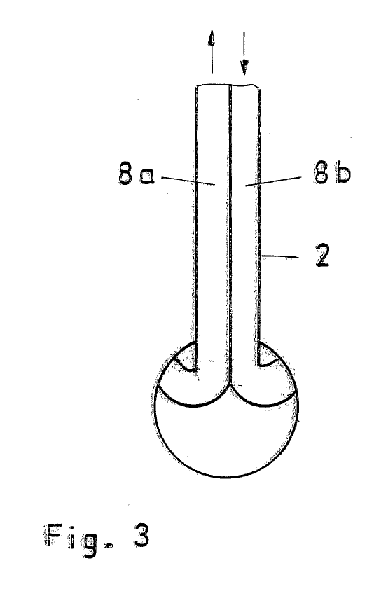

FIG. 3 an electrode with two cavities; and

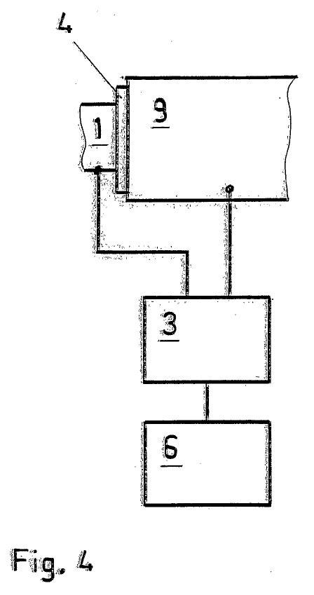

FIG. 4 a device for removing an endoprosthesis with a sleeve as an electrode.

In the following embodiments, the devices and the methods for removing an endoprosthesis will be explained together in more detail.

### FIRST EMBODIMENT

A device for removing an endoprosthesis 1 from biological and/or biocompatible materials 4 as materials surrounding the endoprosthesis 1 is comprised substantially of an electrode 2 and an HF generator 3.

FIG. 1 shows a device for removing an endoprosthesis 1 in a schematic illustration.

The HF generator 3 is/will be connected to the endoprosthesis 1 as conductor and to the electrode 2. The electrode 2 is positioned in a passage 5 that is introduced into the tissue 4 and/or the biomaterial and engages the terminal area of the endoprosthesis 1 so that at the terminal area of the endoprosthesis 1 an electrically conducting connection exists. The endoprosthesis 1 itself is a conductor. The HF generator 3 represents an alternating current source wherein at least the surface area of the endoprosthesis 1 is heated and, as a result of this, the tissue 4 and/or biomaterial becomes detached from the endoprosthesis 1.

The electrode 2 is guided in the passage 5 within the tissue 4 and biomaterial in such a way that the second electrode 2 is arranged at an angle to the endoprosthesis 1 as the first electrode (illustration in FIG. 1).

The terminal area of the electrode 2 as electrode head can be larger in cross-section than the cross-section of the remaining electrode.

The electrical connection is based on the contact between the endoprosthesis 1 as a conductor and the electrode 2 in the area of the electrode head.

The electrode head itself can advantageously be provided in a further embodiment with a spreading mechanisms so that a secure contact between the endoprosthesis 1 as conductor and the electrode head of the electrode 2 can be ensured. The terminal area is advantageously a net and the spreading mechanisms is a balloon within this net.

The HF generator 3 is connected to a data processing device 6 in such a way that the energy introduction through the electrode 2 is realized as a function of the frequency of the high voltage, the geometry of the endoprosthesis 1, and the total electric power.

Simultaneously with or after the HF treatment, the endoprosthesis 1 is coupled to a tool for removing the endoprosthesis.

In a further variant of the first embodiment the data processing device 6 can be connected with the HF generator 3 and a scanner for determining the geometry of the endoprosthesis 1 and/or the adhesion of the endoprosthesis 1 in/on the tissue 4 and biomaterial as a control or governing circuit. The magnitude of energy introduction is controlled as a function of the geometry and/or adhesion between the endoprosthesis 1 as well as the tissue 4 or biomaterial.

### SECOND EMBODIMENT

A device for separating an endoprosthesis 1 as a conductor from tissue 4 and/or biomaterial is comprised substantially of a first electrode 2, a second electrode 7 an HF generator 3.

FIG. 2 shows a device for removing an endoprosthesis with two electrodes 2, 7 positioned at an angle to the endoprosthesis 1 in a schematic illustration.

An area of the endoprosthesis 1 itself is the conductor that is/will be contacted with the electrodes. The first electrode 2 and the second electrode 7 are/will be connected to the HF generator 3 as alternating current source so that at least the surface area of the endoprosthesis 1 as a conductor is heated so that the tissue 4 and/or biomaterial becomes detached from the endoprosthesis 1.

The electrodes 2, 7 are each guided within a passage 5 in the tissue 4 and/or biomaterial as material surrounding the endoprosthesis 1 such that the surface of the endoprosthesis 1 and the first electrode 2 as well as the second electrode 7 are arranged at an angle relative to one another (illustration in FIG. 2).

The electrical connection is based on the contact of the electrodes 2, 7 with the endoprosthesis 1, respectively.

The HF generator 3 is/will be connected to a data processing device 6 such that the energy introduction through the electrodes 2, 7 is realized as a function of the frequency of the high voltage, the geometry of the endoprosthesis 1, and the total electric power.

The endoprosthesis 1 is/will be coupled simultaneously with or after HF treatment to a tool for removing the endoprosthesis 1. This device serves for application of tensile forces, pressure forces, or bending loads.

In a further variant, the data processing device 6 can/will be connected to the HF generator 3 and a scanner for determining the geometry of the endoprosthesis 1 and/or adhesion of the endoprosthesis 1 in/on tissue 4 and biomaterial as a control or governing circuit such that the electrodes 2, 7 and the magnitude of energy introduction is positioned and controlled as a function of adhesion between endoprosthesis 1 and tissue 4 as well as biomaterial.

In a further variant of the first and the second embodiments, the at least one electrode 2 can be embodied as a drill, a chisel, an electrode, or an electrode comprised of two partial electrodes for the biological and biocompatible material 4.

The electrode 2 in a further variant of the embodiments and a plate-shaped electrode are connected either with said HF generator 3 or an HF generator. The plate-shaped electrode can be a grounded contact for the patient or the grounded patient himself.

The partial electrodes of the electrode 2 that are spaced apart from one another are connected in this embodiment either to said HF generator 3 or an HF generator.

In addition, a coil that produces an external magnetic field or a permanent magnet can contribute to removal being controlled in a spatial and temporal fashion.

In accordance with a further variant of the first and the second embodiments, the at least one electrode 2, 7 itself can furthermore comprise also one or several cavities 8a, 8b (illustration of FIG. 3) for a flowable material. For this purpose, at least one of the cavities 8a, 8b is connected to a conveying device for the flowable material.

### THIRD EMBODIMENT

A device for removing an endoprosthesis 1 from tissue 4 and/or biomaterials as a material surrounding the endoprosthesis 1 is comprised substantially of an electrode as a sleeve 9 and an HF generator 3.

FIG. 3 shows a device for removing an endoprosthesis 1 with a sleeve 9 as an electrode in a schematic illustration.

The HF generator 3 is/will be connected to the endoprosthesis 1 as a conductor and the sleeve 9. The sleeve 9 is located in the terminal area on the tissue 4 that surrounds the endoprosthesis 1 and is in the form of bone and/or biomaterial. The endoprosthesis 1 itself is a conductor. The HF generator 3 represents an alternating current source, wherein at least the surface area of the endoprosthesis 1 is heated and, as a result of this, the tissue 4 and/or biomaterial becomes detached from the endoprosthesis 1.

The HF generator 3 is/will be connected to a data processing device 6 in such a way that the energy introduction through the electrode 2 is realized as a function of the frequency of the high voltage, the geometry of the endoprosthesis 1, and the total electric power.

The endoprosthesis 1 is/will be at the same time coupled to a pulling device for removal.

In one variant of the third embodiment the sleeve 9 can be a component of a cooling device wherein the sleeve 9 is provided either with at least one passage for a cooling agent or with Peltier elements.

In a further variant of the embodiments, at least the surgery area can be located at least partially in a Faraday cage wherein this cage is connected to a potential. In this way, the electromagnetic compatibility (EMC) can be ensured. Moreover, by means of a coil or a permanent magnet a movable magnetic field can be built up that controls the location and time of energy introduction into the endoprosthesis or implant.

What is claimed is:

1.-25. (canceled)

26. A device for removing an endoprosthesis or an implant that is implanted in the body and is comprised of a synthetic material, wherein the endoprosthesis, the implant or an area of the endoprosthesis or of the implant is a conductor, the device comprising:
a first electrode, wherein the first electrode is connected to the surface area of the endoprosthesis or the implant in an electrically conducting way or is contacted with an area of the material surrounding the endoprosthesis or the implant:
an HF generator as an alternating current source, wherein the first electrode and said conductor are connected to the HF generator such that energy is introduced into the tissue or biomaterial surrounding the endoprosthesis or the implant causing the tissue or the biomaterial to become detached from the endoprosthesis or the implant; and
a tool for removing the endoprosthesis or the implant, wherein the tool is coupled to the endoprosthesis or the implant, simultaneously with or after the HF processing.

27. The device according to claim 26, wherein the first electrode is guided in a passage in the tissue or the biomaterial and is optionally positioned at an angle relative to the endoprosthesis or to the implant, wherein an electrical connection is caused by contacting with the first electrode the surface of the endoprosthesis or the implant.

28. The device according to claim 26, further comprising a second electrode, wherein the first electrode and the second electrode each are each guided in a passage in the tissue or the biomaterial and are optionally positioned at an angle to the endoprosthesis or to the implant, wherein an electrical connection is caused by contacting with the first and second electrodes the surface of the endoprosthesis or the implant.

29. The device according to claim 28, wherein terminal areas of the first and second electrodes are in the form of a head and wherein the head has a cross-section that is greater than a cross-section of a remaining portion of the first and second electrodes, respectively.

30. The device according to claim 29, wherein the head comprises a spreading mechanism.

31. The device according to claim 28, wherein at least one of the first and second electrodes has at least one cavity and wherein the at least one cavity is connected to a conveying device for flowable materials.

32. The device according to claim 31, wherein the terminal area of at least one of the first and second electrodes has at least one opening and wherein the at least one opening and the cavity are connected to one another.

33. The device according to claim 28, wherein at least one of the first and second electrodes is a cutting or non-cutting tool for the tissue or biomaterial.

34. The device according to claim 26, wherein the first electrode is simultaneously an electrode relative to a plate-shaped electrode and wherein the first electrode and the plate-shaped electrode are connected to the HF generator.

35. The device according to claim 28, wherein at least one of the first and second electrodes is comprised of two spaced-apart partial electrodes that are connected to the HF generator.

36. The device according to claim 26, wherein the first electrode that contacts the at least one area of the material surrounding the endoprosthesis or the implant is a sleeve.

37. The device according to claim 36, wherein the sleeve is a component of a cooling device wherein the sleeve is provided either with at least one passage for a cooling agent or with Peltier elements.

38. The device according to claim 28, wherein at least one of the first and second electrodes has at least one area that is comprised of twisted-together wires of an electrically conductive material.

39. The device according to claim 26, wherein at least the surgery area is partially located within a Faraday cage and wherein the Faraday cage is connected to a potential.

40. The device according to claim 26, wherein the conductor or the at least one area of the material surrounding the endoprosthesis or the implant contacted by at least one additional electrode contacting said material and the first electrode are connected with the HF generator such that at least one surface area of the endoprosthesis or of the implant is acted on by the skin effect.

41. The device according to claim 28, further comprising a data processing device, wherein the HF generator is connected to the data processing device such that the energy is introduced through at least one of the first and second electrodes as a function of the frequency of the high voltage, the geometry of the endoprosthesis or the implant, and the total electric power.

42. The device according to claim 41, further comprising a scanner for determining the geometry of the endoprosthesis or the implant and/or adhesion of the endoprosthesis or the implant in/on the tissue or biomaterial, wherein the data processing device is connected with the HF generator and the scanner so as to form a control or governing circuit such that the magnitude of the energy that is introduced is controlled as a function of the geometry and/or adhesion between the endoprosthesis or the implant as well as the tissue or biomaterial.

43. The device according to claim 41, further comprising a scanner for determining the geometry of the endoprosthesis or the implant and/or adhesion of the endoprosthesis or the implant in/on the tissue or biomaterial, wherein the data processing device is connected with the HF generator and the scanner so as to form a control or governing circuit such that the magnitude of the energy that is introduced and the position of the first and second electrodes is controlled and selected as a function of the geometry and/or adhesion between the endoprosthesis or implant and the tissue or biomaterial.

44. The device according to claim 26, wherein the tool for removing the endoprosthesis or the implant is a device transmitting pulling forces, pressure forces and/or bending moments onto the endoprosthesis or the implant for mechanically assisting the detachment and the removal of the endoprosthesis or the implant.

45. The device according to claim 26, further comprising a coil or a permanent magnet arranged such that a location and a time of snap over of the high frequency current between the tissue or biomaterial and the endoprosthesis or implant is controlled.

46. A method for removal of an endoprosthesis or an implant that is implanted in the body and is comprised of a synthetic material, the method comprising the steps of:
configuring the endoprosthesis, the implant or an area of the endoprosthesis or the implant as a conductor;
connecting a first electrode that is connected to the surface area of the endoprosthesis or the implant in an electrically conducting way or contacted with an area of the material surrounding the endoprosthesis or the implant and the conductor to an HF generator as an alternating current source;
introducing energy into the tissue or biomaterial surrounding the endoprosthesis or the implant with the HF generator causing the tissue or biomaterial to become detached from the endoprosthesis or the implant; and
coupling the endoprosthesis or the implant simultaneously with or after the HF processing to a tool for removing the endoprosthesis or the implant.

47. The method according to claim 46, comprising the step of introducing a passage into the material surrounding the endoprosthesis or the implant and inserting into the passage the first electrode, wherein an electrical connection is provided by contacting with the first electrode the surface of the endoprosthesis or the implant.

48. The method according to claim 46, comprising the step of introducing passages into the material surrounding the endoprosthesis or the implant and inserting into the passages the first electrode and a second electrode, respectively, wherein an electrical connection is provided by contacting with the first and second electrodes the surface of the endoprosthesis or the implant.

49. The method according to claim 46, comprising the step of providing a second electrode in the form of a sleeve, wherein the sleeve is positioned on at least one area of the material surrounding the endoprosthesis or the implant.

50. The method according to claim 46, wherein the endoprosthesis or the implant as a conductor, or at least one area of the material that surrounds the endoprosthesis or the implant by a second electrode contacting the material, and the first electrode are connected with the HF generator such that on at least one surface area of the endoprosthesis or the implant a skin effect is acting.

51. The method according to claim 46, further comprising the step of connecting the HF generator to a data processing device such that energy is introduced by the first electrode and optionally a second electrode as a function of the frequency of the high voltage, the geometry of the endoprosthesis or the implant, and the total electric power.

52. The method according to claim 46, further comprising the step of introducing an electrically conducting liquid into the tissue or biomaterial surrounding the endoprosthesis or the implant.
